(19) United States
(12) Patent Application Publication
(10) Pub. No.: US 2020/0027552 A1
(43) Pub. Date: Jan. 23, 2020

(54) METHOD FOR PREDICTING COMFORTABLE SLEEP BASED ON ARTIFICIAL INTELLIGENCE

(71) Applicant: LG ELECTRONICS INC., Seoul (KR)

(72) Inventor: Myunghee LEE, Seoul (KR)

(73) Assignee: LG ELECTRONICS INC., Seoul (KR)

(21) Appl. No.: 16/588,421

(22) Filed: Sep. 30, 2019

(30) Foreign Application Priority Data
Aug. 21, 2019 (KR) 10-2019-0102376

(51) Int. Cl. G16H 40/67; G06N 20/00; A61B 5/00; A61B 5/11; G06K 9/00

(52) U.S. Cl. CPC G16H 40/67; G06N 20/00; A61B 5/4812; A61B 5/7267; A61B 5/0077; A61B 5/0022; A61B 5/4803; G06K 9/00302; G06K 9/00335; A61B 5/746; A61B 5/11

(57) ABSTRACT

Provided are a method of analyzing sleep and an AI server having a sleep analysis function. The method of analyzing sleep using an AI server includes receiving sleep state data obtained through a monitoring device; determining factors affecting a sleep time by applying the sleep state data to a previously trained sleep analysis model; and determining an appropriate sleep time by applying the factors affecting the sleep time to the previously trained sleep time estimation model. Therefore, by easily estimating an appropriate sleep time of a user, the method can contribute to a user's health promotion. Artificial intelligent device according to the present invention may be linked with an artificial intelligence module, a drone (unmanned aerial vehicle (UAV)), a robot, an augmented reality (AR) device, a virtual reality (VR) device, devices related to 5G services, and the like.

### CROSS-REFERENCE TO RELATED APPLICATION

This application claims the benefit of Korean Patent Application No. 10-2019-0102376 filed on Aug. 21, 2019, the entire contents of which is incorporated herein by reference for all purposes as if fully set forth herein.

### BACKGROUND OF THE INVENTION

### Field of the Invention

The present invention relates to a method of analyzing sleep and an AI server having a sleep analysis function, and more particularly, to a method of analyzing sleep and an AI server having a sleep analysis function that can contribute to a user's health by estimating an appropriate sleep time.

### Related Art

The human body charges energy consumed during the day while sleeping at night. Further, sleep helps to recover a damaged central nervous system and strengthens the body's immunity. Further, dreams may help improve memory through a process of moving memories stored at short-term storage during the day to long-term storage.

Humans have a sleep cycle that sleeps and dreams from a shallow sleep to a deep sleep while sleeping and then back to a shallow sleep and repeats this sleep cycle. In fact, a method of getting a good night's sleep with short sleep may be helped when understanding the sleep cycle. Because the sleep cycle is sometimes determined as a body signal according to a state of a body, when an appropriate sleep time may be estimated, it may help health care.

However, conventional sleep waking devices and methods are to merely set a wake-up time and wake up at a specific time regardless of an appropriate sleep time.

### SUMMARY OF THE INVENTION

An object of the present invention is to solve the above-described needs and/or problems.

The present invention further provides a method of analyzing sleep and an AI server having a sleep analysis function that can contribute to health promotion of a user by estimating an appropriate sleep time required for each person.

The present invention further provides a method of analyzing sleep and an AI server having a sleep analysis function that can automatically set alarm based on an appropriate sleep time.

The present invention further provides a method of analyzing sleep and an AI server having a sleep analysis function that can generate a signal to control lighting during sleep according to a sleep time.

In an aspect, a method of analyzing sleep includes receiving sleep state data obtained through a monitoring device; determining factors affecting a sleep time by applying the sleep state data to a previously trained sleep analysis model; and determining an appropriate sleep time by applying the factors affecting the sleep time to the previously trained sleep time estimation model.

The sleep state data includes at least one of image information, voice information, or past sleep history information including a user.

The monitoring device may include at least one of a camera or a microphone.

The image information may include at least one of facial recognition information before sleep, facial recognition information upon waking up, motion information upon waking up, sleep time information, or time information required to wake-up after alarming.

The voice information may include at least one of loudness information, frequency information, or duration of a voice.

The receiving of sleep state data may include receiving the sleep state data from a 5G network to which the monitoring device is connected.

The sleep analysis model may include at least one of a flush identification model or a wake-up state determination model, wherein the factors affecting the sleep time may include at least one of information on whether the user flushes, facial expression information of the user, behavior pattern information of the user, or sound pattern information determined to yawn by the user.

The determining of factors affecting a sleep time may include applying the sleep state data to the flush identification model; and determining whether the user flushes according to an output value of the flush identification model.

The determining of factors affecting a sleep time may include applying the sleep state data to the wake-up state determination model; and determining the facial expression information or the motion information according to an output value of the wake-up state determination model.

The determining of an appropriate sleep time may include applying factors affecting the sleep time to the sleep time estimation model; and determining the appropriate sleep time according to an output value of the sleep time estimation model.

Information about the appropriate sleep time may include a specific date and the appropriate sleep time of the specific date.

The method may further include generating a signal for controlling an external terminal communicatively connected to the AI server based on the appropriate sleep time.

The controlling signal may be a wake-up alarm signal, wherein the generating of a signal may include checking a sleep entry time of a user based on image information obtained through the monitoring device; determining a wake-up time based on the sleep entry time; and generating the wake-up alarm signal including the wake-up time.

The controlling signal may be a lighting control signal, wherein the generating of a signal may include obtaining outgoing time information of the user through the monitoring device; determining a sleep entry time based on the outgoing time information; and generating a signal to control at least one of a wavelength band or illuminance of light of lighting at the sleep entry time.

In another aspect, an AI server having a sleep analysis function includes a transceiver for receiving sleep state data from an external monitoring device; a processor for applying the sleep state data to a previously trained sleep analysis model to determine factors affecting a sleep time, and applying the factors affecting the sleep time to a previously trained sleep time estimation model to determine an appropriate sleep time.

The effects of a method of analyzing sleep and an AI server having a sleep analysis function according to an embodiment of the present invention are as follows.

The present invention can contribute to a user's health promotion by estimating an appropriate sleep time required for each person.

Further, the present invention can automatically set alarm based on an appropriate sleep time.

Further, the present invention can generate a signal to control lighting during sleep according to a sleep time.

The effects of the present invention are not limited to the above-described effects and the other effects will be understood by those skilled in the art from the following description.

### BRIEF DESCRIPTION OF THE DRAWINGS

The accompany drawings, which are included to provide a further understanding of the present invention and are incorporated on and constitute a part of this specification illustrate embodiments of the present invention and together with the description serve to explain the principles of the present invention.

FIG. 1 illustrates one embodiment of an AI device.

FIG. 2 is a block diagram of a wireless communication system to which methods proposed in the disclosure are applicable.

FIG. 3 is a diagram showing an example of a signal transmission/reception method in a wireless communication system.

FIG. 4 shows an example of basic operations of an autonomous vehicle and a 5G network in a 5G communication system.

FIG. 5 is a block diagram of an AI device according to an embodiment of the present invention.

FIG. 6 is a diagram illustrating a general artificial neural network model.

FIG. 7 illustrates a process of obtaining sleep state data from an external terminal according to an embodiment of the present invention.

FIG. 8 is a diagram illustrating image processing through CNN according to an embodiment of the present invention.

FIG. 9 is a flowchart illustrating a method of estimating an appropriate sleep time according to an embodiment of the present invention.

FIGS. 10 and 11 are diagrams illustrating a method of learning and using a sleep time estimation model according to an embodiment of the present invention.

FIGS. 12 and 13 are diagrams illustrating an alarm setting method according to an embodiment of the present invention.

FIGS. 14 and 15 are diagrams illustrating a light control method according to an embodiment of the present invention.

FIG. 16 is an overall sequence diagram according to an embodiment of the present invention.

### DESCRIPTION OF EXEMPLARY EMBODIMENTS

In what follows, embodiments disclosed in this document will be described in detail with reference to appended drawings, where the same or similar constituent elements are given the same reference number irrespective of their drawing symbols, and repeated descriptions thereof will be omitted. In describing an embodiment disclosed in the present specification, if a constituting element is said to be “connected” or “attached” to other constituting element, it should be understood that the former may be connected or attached directly to the other constituting element, but there may be a case in which another constituting element is present between the two constituting elements. Also, in describing an embodiment disclosed in the present document, if it is determined that a detailed description of a related art incorporated herein unnecessarily obscure the gist of the embodiment, the detailed description thereof will be omitted. Also, it should be understood that the appended drawings are intended only to help understand embodiments disclosed in the present document and do not limit the technical principles and scope of the present invention; rather, it should be understood that the appended drawings include all of the modifications, equivalents or substitutes described by the technical principles and belonging to the technical scope of the present invention.

Terms including an ordinal number such as a “first” and “second” may be used for describing various elements, and the above-described components are not limited by the above terms. The terms are used for distinguishing one constituent element from another constituent element.

When it is described that a constituent element is “connected” or “electrically connected” to another constituent element, the element may be “directly connected” or “directly electrically connected” to the other constituent elements or may be “connected” or “electrically connected” to the other constituent elements through a third element. However, when it is described that an element is “directly connected” or “directly electrically connected” to another element, no element may exist between the element and the other element.

Unless the context otherwise clearly indicates, words used in the singular include the plural, the plural includes the singular.

Further, in the present invention, a term “comprise” or “have” indicates presence of a characteristic, numeral, step, operation, element, component, or combination thereof described in a specification and does not exclude presence or addition of at least one other characteristic, numeral, step, operation, element, component, or combination thereof.

Hereinafter, 5G generation (5th generation mobile communication) required by a device and/or an AI processor requiring AI processed information will be described in paragraph A to paragraph G.

[5G Scenario]

The three main requirement areas in the 5G system are (1) enhanced Mobile Broadband (eMBB) area, (2) massive Machine Type Communication (mMTC) area, and (3) Ultra-Reliable and Low Latency Communication (URLLC) area.

Some use case may require a plurality of areas for optimization, but other use case may focus only one Key Performance Indicator (KPI). The 5G system supports various use cases in a flexible and reliable manner.

eMBB far surpasses the basic mobile Internet access, supports various interactive works, and covers media and entertainment applications in the cloud computing or augmented reality environment. Data is one of core driving elements of the 5G system, which is so abundant that for the first time, the voice-only service may be disappeared. In the 5G, voice is expected to be handled simply by an application program using a data connection provided by the communication system. Primary causes of increased volume of traffic are increase of content size and increase of the number of applications requiring a high data transfer rate. Streaming service (audio and video), interactive video, and mobile Internet connection will be more heavily used as more and more devices are connected to the Internet. These application programs require always-on connectivity to push real-time information and notifications to the user. Cloud-based storage and applications are growing rapidly in the mobile communication platforms, which may be applied to both of business and entertainment uses. And the cloud-based storage is a special use case that drives growth of uplink data transfer rate. The 5G is also used for cloud-based remote works and requires a much shorter end-to-end latency to ensure excellent user experience when a tactile interface is used. Entertainment, for example, cloud-based game and video streaming, is another core element that strengthens the requirement for mobile broadband capability. Entertainment is essential for smartphones and tablets in any place including a high mobility environment such as a train, car, and plane. Another use case is augmented reality for entertainment and information search. Here, augmented reality requires very low latency and instantaneous data transfer.

Also, one of highly expected 5G use cases is the function that connects embedded sensors seamlessly in every possible area, namely the use case based on mMTC. Up to 2020, the number of potential IoT devices is expected to reach 20.4 billion. Industrial IoT is one of key areas where the 5G performs a primary role to maintain infrastructure for smart city, asset tracking, smart utility, agriculture and security.

URLLC includes new services which may transform industry through ultra-reliable/ultra-low latency links, such as remote control of major infrastructure and self-driving cars. The level of reliability and latency are essential for smart grid control, industry automation, robotics, and drone control and coordination.

Next, a plurality of use cases will be described in more detail.

The 5G may complement Fiber-To-The-Home (FTTH) and cable-based broadband (or DOCSIS) as a means to provide a stream estimated to occupy hundreds of megabits per second up to gigabits per second. This fast speed is required not only for virtual reality and augmented reality but also for transferring video with a resolution more than 4K (6K, 8K or more). VR and AR applications almost always include immersive sports games. Specific application programs may require a special network configuration. For example, in the case of VR game, to minimize latency, game service providers may have to integrate a core server with the edge network service of the network operator.

Automobiles are expected to be a new important driving force for the 5G system together with various use cases of mobile communication for vehicles. For example, entertainment for passengers requires high capacity and high mobile broadband at the same time. This is so because users continue to expect a high-quality connection irrespective of their location and moving speed. Another use case in the automotive field is an augmented reality dashboard. The augmented reality dashboard overlays information, which is a perception result of an object in the dark and contains distance to the object and object motion, on what is seen through the front window. In a future, a wireless module enables communication among vehicles, information exchange between a vehicle and supporting infrastructure, and information exchange among a vehicle and other connected devices (for example, devices carried by a pedestrian). A safety system guides alternative courses of driving so that a driver may drive his or her vehicle more safely and to reduce the risk of accident. The next step will be a remotely driven or self-driven vehicle. This step requires highly reliable and highly fast communication between different self-driving vehicles and between a self-driving vehicle and infrastructure. In the future, it is expected that a self-driving vehicle takes care of all of the driving activities while a human driver focuses on dealing with an abnormal driving situation that the self-driving vehicle is unable to recognize. Technical requirements of a self-driving vehicle demand ultra-low latency and ultra-fast reliability up to the level that traffic safety may not be reached by human drivers.

The smart city and smart home, which are regarded as essential to realize a smart society, will be embedded into a high-density wireless sensor network. Distributed networks comprising intelligent sensors may identify conditions for cost-efficient and energy-efficient conditions for maintaining cities and homes. A similar configuration may be applied for each home. Temperature sensors, window and heating controllers, anti-theft alarm devices, and home appliances will be all connected wirelessly. Many of these sensors typified with a low data transfer rate, low power, and low cost. However, for example, real-time HD video may require specific types of devices for the purpose of surveillance.

As consumption and distribution of energy including heat or gas is being highly distributed, automated control of a distributed sensor network is required. A smart grid collects information and interconnect sensors by using digital information and communication technologies so that the distributed sensor network operates according to the collected information. Since the information may include behaviors of energy suppliers and consumers, the smart grid may help improving distribution of fuels such as electricity in terms of efficiency, reliability, economics, production sustainability, and automation. The smart grid may be regarded as a different type of sensor network with a low latency.

The health-care sector has many application programs that may benefit from mobile communication. A communication system may support telemedicine providing a clinical care from a distance. Telemedicine may help reduce barriers to distance and improve access to medical services that are not readily available in remote rural areas. It may also be used to save lives in critical medical and emergency situations. A wireless sensor network based on mobile communication may provide remote monitoring and sensors for parameters such as the heart rate and blood pressure.

Wireless and mobile communication are becoming increasingly important for industrial applications. Cable wiring requires high installation and maintenance costs. Therefore, replacement of cables with reconfigurable wireless links is an attractive opportunity for many industrial applications. However, to exploit the opportunity, the wireless connection is required to function with a latency similar to that in the cable connection, to be reliable and of large capacity, and to be managed in a simple manner. Low latency and very low error probability are new requirements that lead to the introduction of the 5G system.

Logistics and freight tracking are important use cases of mobile communication, which require tracking of an inventory and packages from any place by using location-based information system. The use of logistics and freight tracking typically requires a low data rate but requires large-scale and reliable location information.

The present invention to be described below may be implemented by combining or modifying the respective embodiments to satisfy the aforementioned requirements of the 5G system.

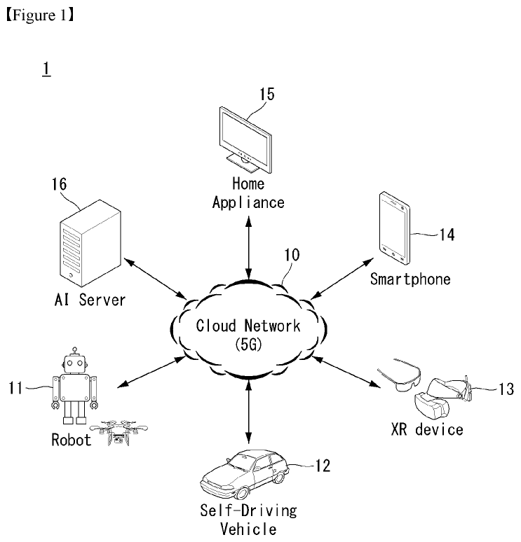

FIG. 1 illustrates one embodiment of an AI device.

Referring to FIG. 1, in the AI system, at least one or more of an AI server 16, robot 11, self-driving vehicle 12, XR device 13, smartphone 14, or home appliance 15 are connected to a cloud network 10. Here, the robot 11, self-driving vehicle 12, XR device 13, smartphone 14, or home appliance 15 to which the AI technology has been applied may be referred to as an AI device (11 to 15).

The cloud network 10 may comprise part of the cloud computing infrastructure or refer to a network existing in the cloud computing infrastructure. Here, the cloud network 10 may be constructed by using the 3G network, 4G or Long Term Evolution (LTE) network, or 5G network.

In other words, individual devices (11 to 16) constituting the AI system may be connected to each other through the cloud network 10. In particular, each individual device (11 to 16) may communicate with each other through the eNB but may communicate directly to each other without relying on the eNB.

The AI server 16 may include a server performing AI processing and a server performing computations on big data.

The AI server 16 may be connected to at least one or more of the robot 11, self-driving vehicle 12, XR device 13, smartphone 14, or home appliance 15, which are AI devices constituting the AI system, through the cloud network 10 and may help at least part of AI processing conducted in the connected AI devices (11 to 15).

At this time, the AI server 16 may teach the artificial neural network according to a machine learning algorithm on behalf of the AI device (11 to 15), directly store the learning model, or transmit the learning model to the AI device (11 to 15).

At this time, the AI server 16 may receive input data from the AI device (11 to 15), infer a result value from the received input data by using the learning model, generate a response or control command based on the inferred result value, and transmit the generated response or control command to the AI device (11 to 15).

Similarly, the AI device (11 to 15) may infer a result value from the input data by employing the learning model directly and generate a response or control command based on the inferred result value.

<AI+Robot>

By employing the AI technology, the robot 11 may be implemented as a guide robot, transport robot, cleaning robot, wearable robot, entertainment robot, pet robot, or unmanned flying robot.

The robot 11 may include a robot control module for controlling its motion, where the robot control module may correspond to a software module or a chip which implements the software module in the form of a hardware device.

The robot 11 may obtain status information of the robot 11, detect (recognize) the surroundings and objects, generate map data, determine a travel path and navigation plan, determine a response to user interaction, or determine motion by using sensor information obtained from various types of sensors.

Here, the robot 11 may use sensor information obtained from at least one or more sensors among lidar, radar, and camera to determine a travel path and navigation plan.

The robot 11 may perform the operations above by using a learning model built on at least one or more artificial neural networks. For example, the robot 11 may recognize the surroundings and objects by using the learning model and determine its motion by using the recognized surroundings or object information. Here, the learning model may be the one trained by the robot 11 itself or trained by an external device such as the AI server 16.

At this time, the robot 11 may perform the operation by generating a result by employing the learning model directly but also perform the operation by transmitting sensor information to an external device such as the AI server 16 and receiving a result generated accordingly.

The robot 11 may determine a travel path and navigation plan by using at least one or more of object information detected from the map data and sensor information or object information obtained from an external device and navigate according to the determined travel path and navigation plan by controlling its locomotion platform.

Map data may include object identification information about various objects disposed in the space in which the robot 11 navigates. For example, the map data may include object identification information about static objects such as wall and doors and movable objects such as a flowerpot and a desk. And the object identification information may include the name, type, distance, location, and so on.

Also, the robot 11 may perform the operation or navigate the space by controlling its locomotion platform based on the control/interaction of the user. At this time, the robot 11 may obtain intention information of the interaction due to the user's motion or voice command and perform an operation by determining a response based on the obtained intention information.

<AI+Autonomous Navigation>

By employing the AI technology, the self-driving vehicle 12 may be implemented as a mobile robot, unmanned ground vehicle, or unmanned aerial vehicle.

The self-driving vehicle 12 may include an autonomous navigation module for controlling its autonomous navigation function, where the autonomous navigation control module may correspond to a software module or a chip which implements the software module in the form of a hardware device. The autonomous navigation control module may be installed inside the self-driving vehicle 12 as a constituting element thereof or may be installed outside the self-driving vehicle 12 as a separate hardware component.

The self-driving vehicle 12 may obtain status information of the self-driving vehicle 12, detect (recognize) the surroundings and objects, generate map data, determine a travel path and navigation plan, or determine motion by using sensor information obtained from various types of sensors.

Like the robot 11, the self-driving vehicle 12 may use sensor information obtained from at least one or more sensors among lidar, radar, and camera to determine a travel path and navigation plan.

In particular, the self-driving vehicle 12 may recognize an occluded area or an area extending over a predetermined distance or objects located across the area by collecting sensor information from external devices or receive recognized information directly from the external devices.

The self-driving vehicle 12 may perform the operations above by using a learning model built on at least one or more artificial neural networks. For example, the self-driving vehicle 12 may recognize the surroundings and objects by using the learning model and determine its navigation route by using the recognized surroundings or object information. Here, the learning model may be the one trained by the self-driving vehicle 12 itself or trained by an external device such as the AI server 16.

At this time, the self-driving vehicle 12 may perform the operation by generating a result by employing the learning model directly but also perform the operation by transmitting sensor information to an external device such as the AI server 16 and receiving a result generated accordingly.

The self-driving vehicle 12 may determine a travel path and navigation plan by using at least one or more of object information detected from the map data and sensor information or object information obtained from an external device and navigate according to the determined travel path and navigation plan by controlling its driving platform.

Map data may include object identification information about various objects disposed in the space (for example, road) in which the self-driving vehicle 12 navigates. For example, the map data may include object identification information about static objects such as streetlights, rocks and buildings and movable objects such as vehicles and pedestrians. And the object identification information may include the name, type, distance, location, and so on.

Also, the self-driving vehicle 12 may perform the operation or navigate the space by controlling its driving platform based on the control/interaction of the user. At this time, the self-driving vehicle 12 may obtain intention information of the interaction due to the user's motion or voice command and perform an operation by determining a response based on the obtained intention information.

<AI+XR>

By employing the AI technology, the XR device 13 may be implemented as a Head-Mounted Display (HMD), Head-Up Display (HUD) installed at the vehicle, TV, mobile phone, smartphone, computer, wearable device, home appliance, digital signage, vehicle, robot with a fixed platform, or mobile robot.

The XR device 13 may obtain information about the surroundings or physical objects by generating position and attribute data about 3D points by analyzing 3D point cloud or image data acquired from various sensors or external devices and output objects in the form of XR objects by rendering the objects for display.

The XR device 13 may perform the operations above by using a learning model built on at least one or more artificial neural networks. For example, the XR device 13 may recognize physical objects from 3D point cloud or image data by using the learning model and provide information corresponding to the recognized physical objects. Here, the learning model may be the one trained by the XR device 13 itself or trained by an external device such as the AI server 16.

At this time, the XR device 13 may perform the operation by generating a result by employing the learning model directly but also perform the operation by transmitting sensor information to an external device such as the AI server 16 and receiving a result generated accordingly.

<AI+Robot+Autonomous Navigation>

By employing the AI and autonomous navigation technologies, the robot 11 may be implemented as a guide robot, transport robot, cleaning robot, wearable robot, entertainment robot, pet robot, or unmanned flying robot.

The robot 11 employing the AI and autonomous navigation technologies may correspond to a robot itself having an autonomous navigation function or a robot 11 interacting with the self-driving vehicle 12.

The robot 11 having the autonomous navigation function may correspond collectively to the devices which may move autonomously along a given path without control of the user or which may move by determining its path autonomously.

The robot 11 and the self-driving vehicle 12 having the autonomous navigation function may use a common sensing method to determine one or more of the travel path or navigation plan. For example, the robot 11 and the self-driving vehicle 12 having the autonomous navigation function may determine one or more of the travel path or navigation plan by using the information sensed through lidar, radar, and camera.

The robot 11 interacting with the self-driving vehicle 12, which exists separately from the self-driving vehicle 12, may be associated with the autonomous navigation function inside or outside the self-driving vehicle 12 or perform an operation associated with the user riding the self-driving vehicle 12.

At this time, the robot 11 interacting with the self-driving vehicle 12 may obtain sensor information in place of the self-driving vehicle 12 and provide the sensed information to the self-driving vehicle 12; or may control or assist the autonomous navigation function of the self-driving vehicle 12 by obtaining sensor information, generating information of the surroundings or object information, and providing the generated information to the self-driving vehicle 12.

Also, the robot 11 interacting with the self-driving vehicle 12 may control the function of the self-driving vehicle 12 by monitoring the user riding the self-driving vehicle 12 or through interaction with the user. For example, if it is determined that the driver is drowsy, the robot 11 may activate the autonomous navigation function of the self-driving vehicle 12 or assist the control of the driving platform of the self-driving vehicle 12. Here, the function of the self-driving vehicle 12 controlled by the robot 12 may include not only the autonomous navigation function but also the navigation system installed inside the self-driving vehicle 12 or the function provided by the audio system of the self-driving vehicle 12.

Also, the robot 11 interacting with the self-driving vehicle 12 may provide information to the self-driving vehicle 12 or assist functions of the self-driving vehicle 12 from the outside of the self-driving vehicle 12. For example, the robot 11 may provide traffic information including traffic sign information to the self-driving vehicle 12 like a smart traffic light or may automatically connect an electric charger to the charging port by interacting with the self-driving vehicle 12 like an automatic electric charger of the electric vehicle.

<AI+Robot+XR>

By employing the AI technology, the robot 11 may be implemented as a guide robot, transport robot, cleaning robot, wearable robot, entertainment robot, pet robot, or unmanned flying robot.

The robot 11 employing the XR technology may correspond to a robot which acts as a control/interaction target in the XR image. In this case, the robot 11 may be distinguished from the XR device 13, both of which may operate in conjunction with each other.

If the robot 11, which acts as a control/interaction target in the XR image, obtains sensor information from the sensors including a camera, the robot 11 or XR device 13 may generate an XR image based on the sensor information, and the XR device 13 may output the generated XR image. And the robot 11 may operate based on the control signal received through the XR device 13 or based on the interaction with the user.

For example, the user may check the XR image corresponding to the viewpoint of the robot 11 associated remotely through an external device such as the XR device 13, modify the navigation path of the robot 11 through interaction, control the operation or navigation of the robot 11, or check the information of nearby objects.

<AI+Autonomous Navigation+XR>

By employing the AI and XR technologies, the self-driving vehicle 12 may be implemented as a mobile robot, unmanned ground vehicle, or unmanned aerial vehicle.

The self-driving vehicle 12 employing the XR technology may correspond to a self-driving vehicle having a means for providing XR images or a self-driving vehicle which acts as a control/interaction target in the XR image. In particular, the self-driving vehicle 12 which acts as a control/interaction target in the XR image may be distinguished from the XR device 13, both of which may operate in conjunction with each other.

The self-driving vehicle 12 having a means for providing XR images may obtain sensor information from sensors including a camera and output XR images generated based on the sensor information obtained. For example, by displaying an XR image through HUD, the self-driving vehicle 12 may provide XR images corresponding to physical objects or image objects to the passenger.

At this time, if an XR object is output on the HUD, at least part of the XR object may be output so as to be overlapped with the physical object at which the passenger gazes. On the other hand, if an XR object is output on a display installed inside the self-driving vehicle 12, at least part of the XR object may be output so as to be overlapped with an image object. For example, the self-driving vehicle 12 may output XR objects corresponding to the objects such as roads, other vehicles, traffic lights, traffic signs, bicycles, pedestrians, and buildings.

If the self-driving vehicle 12, which acts as a control/interaction target in the XR image, obtains sensor information from the sensors including a camera, the self-driving vehicle 12 or XR device 13 may generate an XR image based on the sensor information, and the XR device 13 may output the generated XR image. And the self-driving vehicle 12 may operate based on the control signal received through an external device such as the XR device 13 or based on the interaction with the user.

[Extended Reality Technology]

eXtended Reality (XR) refers to all of Virtual Reality (VR), Augmented Reality (AR), and Mixed Reality (MR). The VR technology provides objects or backgrounds of the real world only in the form of CG images, AR technology provides virtual CG images overlaid on the physical object images, and MR technology employs computer graphics technology to mix and merge virtual objects with the real world.

MR technology is similar to AR technology in a sense that physical objects are displayed together with virtual objects. However, while virtual objects supplement physical objects in the AR, virtual and physical objects co-exist as equivalents in the MR.

The XR technology may be applied to Head-Mounted Display (HMD), Head-Up Display (HUD), mobile phone, tablet PC, laptop computer, desktop computer, TV, digital signage, and so on, where a device employing the XR technology may be called an XR device.

Hereinafter, 5G communication (5th generation mobile communication) required by an apparatus requiring AI processed information and/or an AI processor will be described through paragraphs A through G.

A. Example of Block Diagram of UE and 5G Network

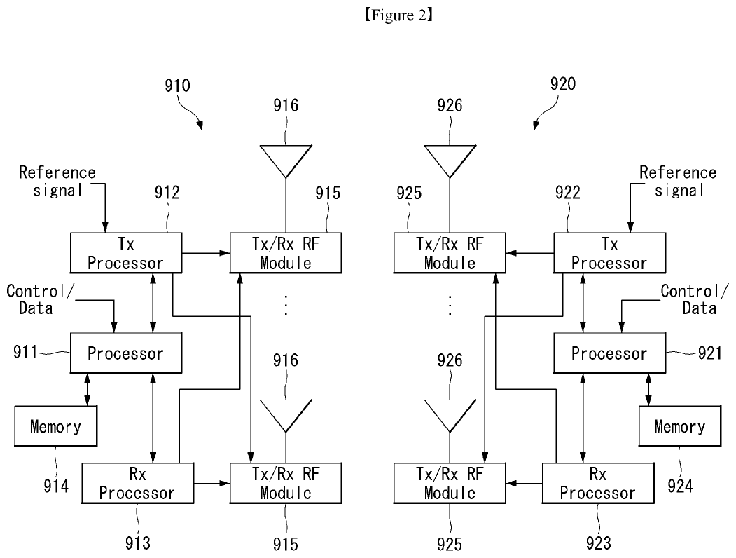

FIG. 2 is a block diagram of a wireless communication system to which methods proposed in the disclosure are applicable.

Referring to FIG. 2, a device (autonomous device) including an autonomous module is defined as a first communication device (910 of FIG. 2), and a processor 911 can perform detailed autonomous operations.

A 5G network including another vehicle communicating with the autonomous device is defined as a second communication device (920 of FIG. 2), and a processor 921 can perform detailed autonomous operations.

The 5G network may be represented as the first communication device and the autonomous device may be represented as the second communication device.

For example, the first communication device or the second communication device may be a base station, a network node, a transmission terminal, a reception terminal, a wireless device, a wireless communication device, an autonomous device, or the like.

For example, the first communication device or the second communication device may be a base station, a network node, a transmission terminal, a reception terminal, a wireless device, a wireless communication device, a vehicle, a vehicle having an autonomous function, a connected car, a drone (Unmanned Aerial Vehicle, UAV), and AI (Artificial Intelligence) module, a robot, an AR (Augmented Reality) device, a VR (Virtual Reality) device, an MR (Mixed Reality) device, a hologram device, a public safety device, an MTC device, an IoT device, a medical device, a Fin Tech device (or financial device), a security device, a climate/environment device, a device associated with 5G services, or other devices associated with the fourth industrial revolution field.

For example, a terminal or user equipment (UE) may include a cellular phone, a smart phone, a laptop computer, a digital broadcast terminal, personal digital assistants (PDAs), a portable multimedia player (PMP), a navigation device, a slate PC, a tablet PC, an ultrabook, a wearable device (e.g., a smartwatch, a smart glass and a head mounted display (HMD)), etc. For example, the HMD may be a display device worn on the head of a user. For example, the HMD may be used to realize VR, AR or MR. For example, the drone may be a flying object that flies by wireless control signals without a person therein. For example, the VR device may include a device that implements objects or backgrounds of a virtual world. For example, the AR device may include a device that connects and implements objects or background of a virtual world to objects, backgrounds, or the like of a real world. For example, the MR device may include a device that unites and implements objects or background of a virtual world to objects, backgrounds, or the like of a real world. For example, the hologram device may include a device that implements 360-degree 3D images by recording and playing 3D information using the interference phenomenon of light that is generated by two lasers meeting each other which is called holography. For example, the public safety device may include an image repeater or an imaging device that can be worn on the body of a user. For example, the MTC device and the IoT device may be devices that do not require direct interference or operation by a person. For example, the MTC device and the IoT device may include a smart meter, a bending machine, a thermometer, a smart bulb, a door lock, various sensors, or the like. For example, the medical device may be a device that is used to diagnose, treat, attenuate, remove, or prevent diseases. For example, the medical device may be a device that is used to diagnose, treat, attenuate, or correct injuries or disorders. For example, the medial device may be a device that is used to examine, replace, or change structures or functions. For example, the medical device may be a device that is used to control pregnancy. For example, the medical device may include a device for medical treatment, a device for operations, a device for (external) diagnose, a hearing aid, an operation device, or the like. For example, the security device may be a device that is installed to prevent a danger that is likely to occur and to keep safety. For example, the security device may be a camera, a CCTV, a recorder, a black box, or the like. For example, the Fin Tech device may be a device that can provide financial services such as mobile payment.

Referring to FIG. 2, the first communication device 910 and the second communication device 920 include processors 911 and 921, memories 914 and 924, one or more Tx/Rx radio frequency (RF) modules 915 and 925, Tx processors 912 and 922, Rx processors 913 and 923, and antennas 916 and 926. The Tx/Rx module is also referred to as a transceiver. Each Tx/Rx module 915 transmits a signal through each antenna 926. The processor implements the aforementioned functions, processes and/or methods. The processor 921 may be related to the memory 924 that stores program code and data. The memory may be referred to as a computer-readable medium. More specifically, the Tx processor 912 implements various signal processing functions with respect to L1 (i.e., physical layer) in DL (communication from the first communication device to the second communication device). The Rx processor implements various signal processing functions of L1 (i.e., physical layer).

UL (communication from the second communication device to the first communication device) is processed in the first communication device 910 in a way similar to that described in association with a receiver function in the second communication device 920. Each Tx/Rx module 925 receives a signal through each antenna 926. Each Tx/Rx module provides RF carriers and information to the Rx processor 923. The processor 921 may be related to the memory 924 that stores program code and data. The memory may be referred to as a computer-readable medium.

B. Signal Transmission/Reception Method in Wireless Communication System

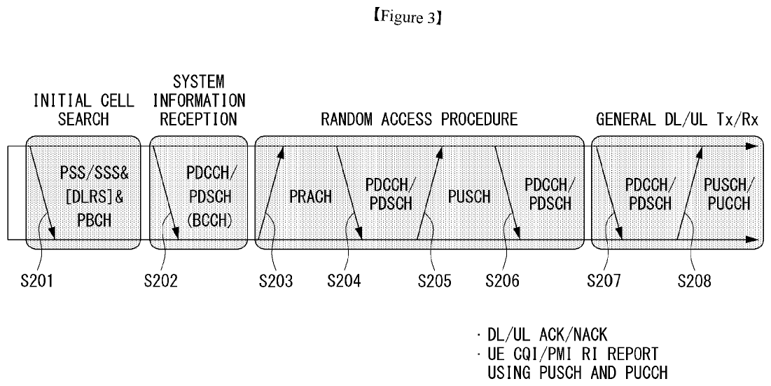

FIG. 3 is a diagram showing an example of a signal transmission/reception method in a wireless communication system.

Referring to FIG. 3, when a UE is powered on or enters a new cell, the UE performs an initial cell search operation such as synchronization with a BS (S201). For this operation, the UE can receive a primary synchronization channel (P-SCH) and a secondary synchronization channel (S-SCH) from the BS to synchronize with the BS and acquire information such as a cell ID. In LTE and NR systems, the P-SCH and S-SCH are respectively called a primary synchronization signal (PSS) and a secondary synchronization signal (SSS). After initial cell search, the UE can acquire broadcast information in the cell by receiving a physical broadcast channel (PBCH) from the BS. Further, the UE can receive a downlink reference signal (DL RS) in the initial cell search step to check a downlink channel state. After initial cell search, the UE can acquire more detailed system information by receiving a physical downlink shared channel (PDSCH) according to a physical downlink control channel (PDCCH) and information included in the PDCCH (S202).

Meanwhile, when the UE initially accesses the BS or has no radio resource for signal transmission, the UE can perform a random access procedure (RACH) for the BS (steps S203 to S206). To this end, the UE can transmit a specificC sequence as a preamble through a physical random access channel (PRACH) (S203 and S205) and receive a random access response (RAR) message for the preamble through a PDCCH and a corresponding PDSCH (S204 and S206). In the case of a contention-based RACH, a contention resolution procedure may be additionally performed.

After the UE performs the above-described process, the UE can perform PDCCH/PDSCH reception (S207) and physical uplink shared channel (PUSCH)/physical uplink control channel (PUCCH) transmission (S208) as normal uplink/downlink signal transmission processes. Particularly, the UE receives downlink control information (DCI) through the PDCCH. The UE monitors a set of PDCCH candidates in monitoring occasions set for one or more control element sets (CORESET) on a serving cell according to corresponding search space configurations. A set of PDCCH candidates to be monitored by the UE is defined in terms of search space sets, and a search space set may be a common search space set or a UE-specific search space set. CORESET includes a set of (physical) resource blocks having a duration of one to three OFDM symbols. A network can configure the UE such that the UE has a plurality of CORESETs. The UE monitors PDCCH candidates in one or more search space sets. Here, monitoring means attempting decoding of PDCCH candidate(s) in a search space. When the UE has successfully decoded one of PDCCH candidates in a search space, the UE determines that a PDCCH has been detected from the PDCCH candidate and performs PDSCH reception or PUSCH transmission on the basis of DCI in the detected PDCCH. The PDCCH can be used to schedule DL transmissions over a PDSCH and UL transmissions over a PUSCH. Here, the DCI in the PDCCH includes downlink assignment (i.e., downlink grant (DL grant)) related to a physical downlink shared channel and including at least a modulation and coding format and resource allocation information, or an uplink grant (UL grant) related to a physical uplink shared channel and including a modulation and coding format and resource allocation information.

An initial access (IA) procedure in a 5G communication system will be additionally described with reference to FIG. 3.

The UE can perform cell search, system information acquisition, beam alignment for initial access, and DL measurement on the basis of an SSB. The SSB is interchangeably used with a synchronization signal/physical broadcast channel (SS/PBCH) block.

The SSB includes a PSS, an SSS and a PBCH. The SSB is configured in four consecutive OFDM symbols, and a PSS, a PBCH, an SSS/PBCH or a PBCH is transmitted for each OFDM symbol. Each of the PSS and the SSS includes one OFDM symbol and 127 subcarriers, and the PBCH includes 3 OFDM symbols and 576 subcarriers.

Cell search refers to a process in which a UE acquires time/frequency synchronization of a cell and detects a cell identifier (ID) (e.g., physical layer cell ID (PCI)) of the cell. The PSS is used to detect a cell ID in a cell ID group and the SSS is used to detect a cell ID group. The PBCH is used to detect an SSB (time) index and a half-frame.

There are 336 cell ID groups and there are 3 cell IDs per cell ID group. A total of 1008 cell IDs are present. Information on a cell ID group to which a cell ID of a cell belongs is provided/acquired through an SSS of the cell, and information on the cell ID among 336 cell ID groups is provided/acquired through a PSS.

The SSB is periodically transmitted in accordance with SSB periodicity. A default SSB periodicity assumed by a UE during initial cell search is defined as 20 ms. After cell access, the SSB periodicity can be set to one of {5 ms, 10 ms, 20 ms, 40 ms, 80 ms, 160 ms} by a network (e.g., a BS).

Next, acquisition of system information (SI) will be described.

SI is divided into a master information block (MIB) and a plurality of system information blocks (SIBs). SI other than the MIB may be referred to as remaining minimum system information. The MIB includes information/parameter for monitoring a PDCCH that schedules a PDSCH carrying SIB1 (SystemInformationBlock1) and is transmitted by a BS through a PBCH of an SSB. SIB1 includes information related to availability and scheduling (e.g., transmission periodicity and SI-window size) of the remaining SIBs (hereinafter, SIBx, xis an integer equal to or greater than 2). SiBx is included in an SI message and transmitted over a PDSCH. Each SI message is transmitted within a periodically generated time window (i.e., SI-window).

A random access (RA) procedure in a 5G communication system will be additionally described with reference to FIG. 3.

A random access procedure is used for various purposes. For example, the random access procedure can be used for network initial access, handover, and UE-triggered UL data transmission. A UE can acquire UL synchronization and UL transmission resources through the random access procedure. The random access procedure is classified into a contention-based random access procedure and a contention-free random access procedure. A detailed procedure for the contention-based random access procedure is as follows.

A UE can transmit a random access preamble through a PRACH as Msg1 of a random access procedure in UL. Random access preamble sequences having different two lengths are supported. A long sequence length 839 is applied to subcarrier spacings of 1.25 kHz and 5 kHz and a short sequence length 139 is applied to subcarrier spacings of 15 kHz, 30 kHz, 60 kHz and 120 kHz.

When a BS receives the random access preamble from the UE, the BS transmits a random access response (RAR) message (Msg2) to the UE. A PDCCH that schedules a PDSCH carrying a RAR is CRC masked by a random access (RA) radio network temporary identifier (RNTI) (RA-RNTI) and transmitted. Upon detection of the PDCCH masked by the RA-RNTI, the UE can receive a RAR from the PDSCH scheduled by DCI carried by the PDCCH. The UE checks whether the RAR includes random access response information with respect to the preamble transmitted by the UE, that is, Msg1. Presence or absence of random access information with respect to Msg1 transmitted by the UE can be determined according to presence or absence of a random access preamble ID with respect to the preamble transmitted by the UE. If there is no response to Msg1, the UE can retransmit the RACH preamble less than a predetermined number of times while performing power ramping. The UE calculates PRACH transmission power for preamble retransmission on the basis of most recent pathloss and a power ramping counter.

The UE can perform UL transmission through Msg3 of the random access procedure over a physical uplink shared channel on the basis of the random access response information. Msg3 can include an RRC connection request and a UE ID. The network can transmit Msg4 as a response to Msg3, and Msg4 can be handled as a contention resolution message on DL. The UE can enter an RRC connected state by receiving Msg4.

C. Beam Management (BM) Procedure of 5G Communication System

A BM procedure can be divided into (1) a DL MB procedure using an SSB or a CSI-RS and (2) a UL BM procedure using a sounding reference signal (SRS). In addition, each BM procedure can include Tx beam swiping for determining a Tx beam and Rx beam swiping for determining an Rx beam.

The DL BM procedure using an SSB will be described.

Configuration of a beam report using an SSB is performed when channel state information (CSI)/beam is configured in RRC_CONNECTED.

A UE receives a CSI-ResourceConfig IE including CSI-SSB-ResourceSetList for SSB resources used for BM from a BS. The RRC parameter “csi-SSB-ResourceSetList” represents a list of SSB resources used for beam management and report in one resource set. Here, an SSB resource set can be set as {SSBx1, SSBx2, SSBx3, SSBx4, . . . }. An SSB index can be defined in the range of 0 to 63.

The UE receives the signals on SSB resources from the BS on the basis of the CSI-SSB-ResourceSetList.

When CSI-RS reportConfig with respect to a report on SSBRI and reference signal received power (RSRP) is set, the UE reports the best SSBRI and RSRP corresponding thereto to the BS. For example, when reportQuantity of the CSI-RS reportConfig IE is set to ‘ssb-Index-RSRP’, the UE reports the best SSBRI and RSRP corresponding thereto to the BS.

When a CSI-RS resource is configured in the same OFDM symbols as an SSB and ‘QCL-TypeD’ is applicable, the UE can assume that the CSI-RS and the SSB are quasi co-located (QCL) from the viewpoint of ‘QCL-TypeD’. Here, QCL-TypeD may mean that antenna ports are quasi co-located from the viewpoint of a spatial Rx parameter. When the UE receives signals of a plurality of DL antenna ports in a QCL-TypeD relationship, the same Rx beam can be applied.

Next, a DL BM procedure using a CSI-RS will be described.

An Rx beam determination (or refinement) procedure of a UE and a Tx beam swiping procedure of a BS using a CSI-RS will be sequentially described. A repetition parameter is set to ‘ON’ in the Rx beam determination procedure of a UE and set to ‘OFF’ in the Tx beam swiping procedure of a BS.

First, the Rx beam determination procedure of a UE will be described.

The UE receives an NZP CSI-RS resource set IE including an RRC parameter with respect to ‘repetition’ from a BS through RRC signaling. Here, the RRC parameter ‘repetition’ is set to ‘ON’.

The UE repeatedly receives signals on resources in a CSI-RS resource set in which the RRC parameter ‘repetition’ is set to ‘ON’ in different OFDM symbols through the same Tx beam (or DL spatial domain transmission filters) of the BS.

The UE determines an RX beam thereof.

The UE skips a CSI report. That is, the UE can skip a CSI report when the RRC parameter ‘repetition’ is set to ‘ON’.

Next, the Tx beam determination procedure of a BS will be described.

A UE receives an NZP CSI-RS resource set IE including an RRC parameter with respect to ‘repetition’ from the BS through RRC signaling. Here, the RRC parameter ‘repetition’ is related to the Tx beam swiping procedure of the BS when set to ‘OFF’.

The UE receives signals on resources in a CSI-RS resource set in which the RRC parameter ‘repetition’ is set to ‘OFF’ in different DL spatial domain transmission filters of the BS.

The UE selects (or determines) a best beam.

The UE reports an ID (e.g., CRI) of the selected beam and related quality information (e.g., RSRP) to the BS. That is, when a CSI-RS is transmitted for BM, the UE reports a CRI and RSRP with respect thereto to the BS.

Next, the UL BM procedure using an SRS will be described.

A UE receives RRC signaling (e.g., SRS-Config IE) including a (RRC parameter) purpose parameter set to ‘beam management” from a BS. The SRS-Config IE is used to set SRS transmission. The SRS-Config IE includes a list of SRS-Resources and a list of SRS-ResourceSets. Each SRS resource set refers to a set of SRS-resources.

The UE determines Tx beamforming for SRS resources to be transmitted on the basis of SRS-SpatialRelation Info included in the SRS-Config IE. Here, SRS-SpatialRelation Info is set for each SRS resource and indicates whether the same beamforming as that used for an SSB, a CSI-RS or an SRS will be applied for each SRS resource.

When SRS-SpatialRelationInfo is set for SRS resources, the same beamforming as that used for the SSB, CSI-RS or SRS is applied. However, when SRS-SpatialRelationInfo is not set for SRS resources, the UE arbitrarily determines Tx beamforming and transmits an SRS through the determined Tx beamforming.

Next, a beam failure recovery (BFR) procedure will be described.

In a beamformed system, radio link failure (RLF) may frequently occur due to rotation, movement or beamforming blockage of a UE. Accordingly, NR supports BFR in order to prevent frequent occurrence of RLF. BFR is similar to a radio link failure recovery procedure and can be supported when a UE knows new candidate beams. For beam failure detection, a BS configures beam failure detection reference signals for a UE, and the UE declares beam failure when the number of beam failure indications from the physical layer of the UE reaches a threshold set through RRC signaling within a period set through RRC signaling of the BS. After beam failure detection, the UE triggers beam failure recovery by initiating a random access procedure in a PCell and performs beam failure recovery by selecting a suitable beam. (When the BS provides dedicated random access resources for certain beams, these are prioritized by the UE). Completion of the aforementioned random access procedure is regarded as completion of beam failure recovery.

D. URLLC (Ultra-Reliable and Low Latency Communication)

URLLC transmission defined in NR can refer to (1) a relatively low traffic size, (2) a relatively low arrival rate, (3) extremely low latency requirements (e.g., 0.5 and 1 ms), (4) relatively short transmission duration (e.g., 2 OFDM symbols), (5) urgent services/messages, etc. In the case of UL, transmission of traffic of a specific type (e.g., URLLC) needs to be multiplexed with another transmission (e.g., eMBB) scheduled in advance in order to satisfy more stringent latency requirements. In this regard, a method of providing information indicating preemption of specific resources to a UE scheduled in advance and allowing a URLLC UE to use the resources for UL transmission is provided.

NR supports dynamic resource sharing between eMBB and URLLC. eMBB and URLLC services can be scheduled on non-overlapping time/frequency resources, and URLLC transmission can occur in resources scheduled for ongoing eMBB traffic. An eMBB UE may not ascertain whether PDSCH transmission of the corresponding UE has been partially punctured and the UE may not decode a PDSCH due to corrupted coded bits. In view of this, NR provides a preemption indication. The preemption indication may also be referred to as an interrupted transmission indication.

With regard to the preemption indication, a UE receives DownlinkPreemption IE through RRC signaling from a BS. When the UE is provided with DownlinkPreemption IE, the UE is configured with INT-RNTI provided by a parameter int-RNTI in DownlinkPreemption IE for monitoring of a PDCCH that conveys DCI format 2_1. The UE is additionally configured with a corresponding set of positions for fields in DCI format 2_1 according to a set of serving cells and positionInDCl by INT-ConfigurationPerServing Cell including a set of serving cell indexes provided by servingCellID, configured having an information payload size for DCI format 2_1 according to dci-Payloadsize, and configured with indication granularity of time-frequency resources according to timeFrequencySect.

The UE receives DCI format 2_1 from the BS on the basis of the DownlinkPreemption IE.

When the UE detects DCI format 2_1 for a serving cell in a configured set of serving cells, the UE can assume that there is no transmission to the UE in PRBs and symbols indicated by the DCI format 2_1 in a set of PRBs and a set of symbols in a last monitoring period before a monitoring period to which the DCI format 2_1 belongs. For example, the UE assumes that a signal in a time-frequency resource indicated according to preemption is not DL transmission scheduled therefor and decodes data on the basis of signals received in the remaining resource region.

E. mMTC (Massive MTC)

mMTC (massive Machine Type Communication) is one of 5G scenarios for supporting a hyper-connection service providing simultaneous communication with a large number of UEs. In this environment, a UE intermittently performs communication with a very low speed and mobility. Accordingly, a main goal of mMTC is operating a UE for a long time at a low cost. With respect to mMTC, 3GPP deals with MTC and NB (NarrowBand)-IoT.

mMTC has features such as repetitive transmission of a PDCCH, a PUCCH, a PDSCH (physical downlink shared channel), a PUSCH, etc., frequency hopping, retuning, and a guard period.

That is, a PUSCH (or a PUCCH (particularly, a long PUCCH) or a PRACH) including specific information and a PDSCH (or a PDCCH) including a response to the specific information are repeatedly transmitted. Repetitive transmission is performed through frequency hopping, and for repetitive transmission, (RF) retuning from a first frequency resource to a second frequency resource is performed in a guard period and the specific information and the response to the specific information can be transmitted/received through a narrowband (e.g., 6 resource blocks (RBs) or 1 RB).

F. Basic Operation Between Autonomous Vehicles Using 5G Communication

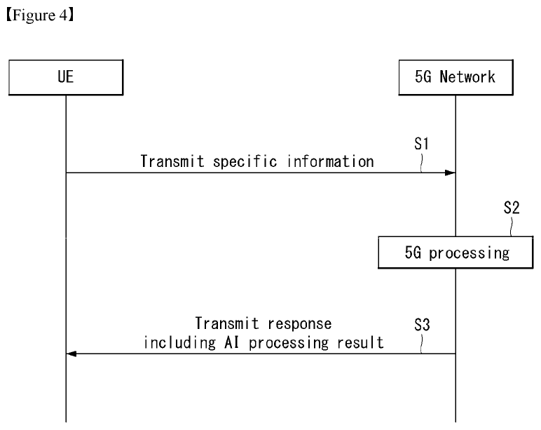

FIG. 4 shows an example of basic operations of an autonomous vehicle and a 5G network in a 5G communication system.

The autonomous vehicle transmits specific information to the 5G network (S1). The specific information may include autonomous driving related information. In addition, the 5G network can determine whether to remotely control the vehicle (S2). Here, the 5G network may include a server or a module which performs remote control related to autonomous driving. In addition, the 5G network can transmit information (or signal) related to remote control to the autonomous vehicle (S3).

G. Applied Operations Between Autonomous Vehicle and 5G Network in 5G Communication System

Hereinafter, the operation of an autonomous vehicle using 5G communication will be described in more detail with reference to wireless communication technology (BM procedure, URLLC, mMTC, etc.) described in FIGS. 1 and 2.

First, a basic procedure of an applied operation to which a method proposed by the present invention which will be described later and eMBB of 5G communication are applied will be described.

As in steps S1 and S3 of FIG. 4, the autonomous vehicle performs an initial access procedure and a random access procedure with the 5G network prior to step S1 of FIG. 4 in order to transmit/receive signals, information and the like to/from the 5G network.

More specifically, the autonomous vehicle performs an initial access procedure with the 5G network on the basis of an SSB in order to acquire DL synchronization and system information. A beam management (BM) procedure and a beam failure recovery procedure may be added in the initial access procedure, and quasi-co-location (QCL) relation may be added in a process in which the autonomous vehicle receives a signal from the 5G network.

In addition, the autonomous vehicle performs a random access procedure with the 5G network for UL synchronization acquisition and/or UL transmission. The 5G network can transmit, to the autonomous vehicle, a UL grant for scheduling transmission of specific information. Accordingly, the autonomous vehicle transmits the specific information to the 5G network on the basis of the UL grant. In addition, the 5G network transmits, to the autonomous vehicle, a DL grant for scheduling transmission of 5G processing results with respect to the specific information. Accordingly, the 5G network can transmit, to the autonomous vehicle, information (or a signal) related to remote control on the basis of the DL grant.

Next, a basic procedure of an applied operation to which a method proposed by the present invention which will be described later and URLLC of 5G communication are applied will be described.

As described above, an autonomous vehicle can receive DownlinkPreemption IE from the 5G network after the autonomous vehicle performs an initial access procedure and/or a random access procedure with the 5G network. Then, the autonomous vehicle receives DCI format 2_1 including a preemption indication from the 5G network on the basis of DownlinkPreemption IE. The autonomous vehicle does not perform (or expect or assume) reception of eMBB data in resources (PRBs and/or OFDM symbols) indicated by the preemption indication. Thereafter, when the autonomous vehicle needs to transmit specific information, the autonomous vehicle can receive a UL grant from the 5G network.

Next, a basic procedure of an applied operation to which a method proposed by the present invention which will be described later and mMTC of 5G communication are applied will be described.

Description will focus on parts in the steps of FIG. 4 which are changed according to application of mMTC.

In step S1 of FIG. 4, the autonomous vehicle receives a UL grant from the 5G network in order to transmit specific information to the 5G network. Here, the UL grant may include information on the number of repetitions of transmission of the specific information and the specific information may be repeatedly transmitted on the basis of the information on the number of repetitions. That is, the autonomous vehicle transmits the specific information to the 5G network on the basis of the UL grant. Repetitive transmission of the specific information may be performed through frequency hopping, the first transmission of the specific information may be performed in a first frequency resource, and the second transmission of the specific information may be performed in a second frequency resource. The specific information can be transmitted through a narrowband of 6 resource blocks (RBs) or 1 RB.

The above-described 5G communication technology can be combined with methods proposed in the present invention which will be described later and applied or can complement the methods proposed in the present invention to make technical features of the methods concrete and clear.

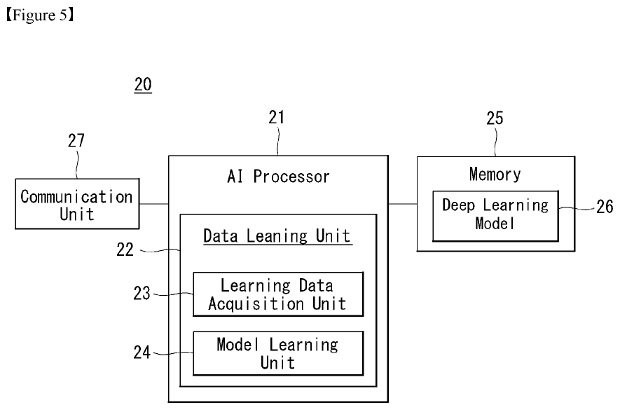

FIG. 5 is a block diagram of an AI device according to an embodiment of the present invention.

An AI device 20 may include an electronic device including an AI module that can perform AI processing, a server including the AI module, or the like. Further, the robot 11, self-driving vehicle 12, XR device 13, smartphone 14, or home appliance 15 to which the AI technology has been applied may be referred to as an AI device (11 to 15)

The AI device 20 may include an AI processor 21, a memory 25, and/or a communication unit 27.

The AI device 20, which is a computing device that can learn a neural network, may be implemented as various electronic devices such as a server, a desktop PC, a notebook PC, and a tablet PC.

The AI processor 21 can learn a neural network using programs stored in the memory 25. In particular, the AI processor 21 can learn a neural network for recognizing data related to vehicles. Here, the neural network for recognizing data related to vehicles may be designed to simulate the brain structure of human on a computer and may include a plurality of network nodes having weights and simulating the neurons of human neural network. The plurality of network nodes can transmit and receive data in accordance with each connection relationship to simulate the synaptic activity of neurons in which neurons transmit and receive signals through synapses. Here, the neural network may include a deep learning model developed from a neural network model. In the deep learning model, a plurality of network nodes is positioned in different layers and can transmit and receive data in accordance with a convolution connection relationship. The neural network, for example, includes various deep learning techniques such as deep neural networks (DNN), convolutional deep neural networks (CNN), recurrent neural networks (RNN), a restricted boltzmann machine (RBM), deep belief networks (DBN), and a deep Q-network, and can be applied to fields such as computer vision, voice recognition, natural language processing, and voice/signal processing.

Meanwhile, a processor that performs the functions described above may be a general purpose processor (e.g., a CPU), but may be an AI-only processor (e.g., a GPU) for artificial intelligence learning.

The memory 25 can store various programs and data for the operation of the AI device 20. The memory 25 may be a nonvolatile memory, a volatile memory, a flash-memory, a hard disk drive (HDD), a solid state drive (SDD), or the like. The memory 25 is accessed by the AI processor 21 and reading-out/recording/correcting/deleting/updating, etc. of data by the AI processor 21 can be performed. Further, the memory 25 can store a neural network model (e.g., a deep learning model 26) generated through a learning algorithm for data classification/recognition according to an embodiment of the present invention.

Meanwhile, the AI processor 21 may include a data learning unit 22 that learns a neural network for data classification/recognition. The data learning unit 22 can learn references about what learning data are used and how to classify and recognize data using the learning data in order to determine data classification/recognition. The data learning unit 22 can learn a deep learning model by acquiring learning data to be used for learning and by applying the acquired learning data to the deep learning model.

The data learning unit 22 may be manufactured in the type of at least one hardware chip and mounted on the AI device 20. For example, the data learning unit 22 may be manufactured in a hardware chip type only for artificial intelligence, and may be manufactured as a part of a general purpose processor (CPU) or a graphics processing unit (GPU) and mounted on the AI device 20. Further, the data learning unit 22 may be implemented as a software module. When the data leaning unit 22 is implemented as a software module (or a program module including instructions), the software module may be stored in non-transitory computer readable media that can be read through a computer. In this case, at least one software module may be provided by an OS (operating system) or may be provided by an application.

The data learning unit 22 may include a learning data acquiring unit 23 and a model learning unit 24.

The learning data acquiring unit 23 can acquire learning data required for a neural network model for classifying and recognizing data. For example, the learning data acquiring unit 23 can acquire, as learning data, vehicle data and/or sample data to be input to a neural network model.

The model learning unit 24 can perform learning such that a neural network model has a determination reference about how to classify predetermined data, using the acquired learning data. In this case, the model learning unit 24 can train a neural network model through supervised learning that uses at least some of learning data as a determination reference. Alternatively, the model learning data 24 can train a neural network model through unsupervised learning that finds out a determination reference by performing learning by itself using learning data without supervision. Further, the model learning unit 24 can train a neural network model through reinforcement learning using feedback about whether the result of situation determination according to learning is correct. Further, the model learning unit 24 can train a neural network model using a learning algorithm including error back-propagation or gradient decent.

When a neural network model is learned, the model learning unit 24 can store the learned neural network model in the memory. The model learning unit 24 may store the learned neural network model in the memory of a server connected with the AI device 20 through a wire or wireless network.

The data learning unit 22 may further include a learning data preprocessor (not shown) and a learning data selector (not shown) to improve the analysis result of a recognition model or reduce resources or time for generating a recognition model.

The learning data preprocessor can preprocess acquired data such that the acquired data can be used in learning for situation determination. For example, the learning data preprocessor can process acquired data in a predetermined format such that the model learning unit 24 can use learning data acquired for learning for image recognition.

Further, the learning data selector can select data for learning from the learning data acquired by the learning data acquiring unit 23 or the learning data preprocessed by the preprocessor. The selected learning data can be provided to the model learning unit 24.

Further, the data learning unit 22 may further include a model estimator (not shown) to improve the analysis result of a neural network model.

The model estimator inputs estimation data to a neural network model, and when an analysis result output from the estimation data does not satisfy a predetermined reference, it can make the model learning unit 22 perform learning again. In this case, the estimation data may be data defined in advance for estimating a recognition model. For example, when the number or ratio of estimation data with an incorrect analysis result of the analysis result of a recognition model learned with respect to estimation data exceeds a predetermined threshold, the model estimator can estimate that a predetermined reference is not satisfied.

The communication unit 27 can transmit the AI processing result by the AI processor 21 to an external electronic device. For example, external electronic devices may include Bluetooth device, autonomous vehicle, robot, drone, AR device, mobile device or home appliance. The communication unit 27 includes transmitter, receiver, and transceiver.

Meanwhile, the AI device 20 shown in FIG. 5 was functionally separately described into the AI processor 21, the memory 25, the communication unit 27, etc., but it should be noted that the aforementioned components may be integrated in one module and referred to as an AI module.

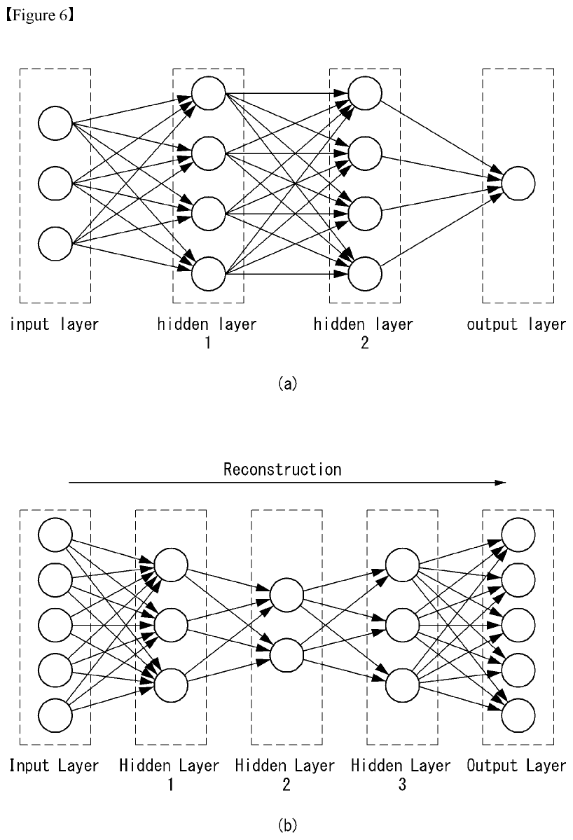

FIG. 6 is a diagram illustrating a general artificial neural network model.

Specifically, FIG. 6(a) is a diagram illustrating a general structure of an artificial neural network, and FIG. 6(b) is a diagram illustrating an autoencoder that performs decoding after encoding and that performs a reconstruction step among artificial neural networks.

The artificial neural network is generally configured with an input layer, a hidden layer, and an output layer, and neurons included in each layer may be connected through a weight. Through a linear combination of a weight and a neuron value and a nonlinear activation function, the artificial neural network may have a form to approximate complex functions. A learning purpose of the artificial neural network is to find a weight that minimizes the difference between an output computed at the output layer and an actual output.

The deep neural network may mean an artificial neural network configured with several hidden layers between an input layer and an output layer. By using many hidden layers, complex nonlinear relationships may be modeled, and a neural network structure that enables advanced abstraction by increasing the number of layers in this way is referred to as deep learning. Deep learning learns a very large amount of data, and when new data is input, deep learning may select a highest probability answer based on the learning results. Therefore, deep learning may operate adaptively according to an input and automatically find characteristic factors in a process of learning a model based on data.

A deep learning-based model may include various deep learning techniques such as deep neural networks (DNN), convolutional deep neural networks (CNN), Recurrent Boltzmann Machine (RNN), Restricted Boltzmann Machine (RBM), deep belief networks (DBN), and a deep Q-network of FIG. 5, but it is not limited thereto. Further, the deep learning-based model may include a machine learning method other than deep learning. For example, a deep learning-based model may be applied to extract a characteristic of input data, and a machine learning-based model may be applied to classify or recognize the input data based on the extracted characteristic. The machine learning-based model may include a support vector machine (SVM), AdaBoost, and the like, but it is not limited thereto.

Referring to FIG. 6(a), the artificial neural network model according to an embodiment of the present invention may include an input layer, a hidden layer, an output layer, and a weight. For example, FIG. 6(a) illustrates a structure of an artificial neural network in which a size of an input layer is 3 and in which a size of first and second hidden layers is 4 and in which a size of an output layer is 1. Specifically, neurons included in the hidden layer may be connected in a linear combination with neurons included in the input layer and individual weights included in the weight. Neurons included in the output layer may be connected by linear combination of neurons included in the hidden layer and individual weights included in the weight. The artificial neural network may find a model that minimizes the difference between an output calculated at the output layer and an actual output.

Further, the artificial neural network according to an embodiment of the present invention may have an artificial neural network structure in which an input layer size is 10 and in which an output layer size is 4 and that does not limit a size of the hidden layer.

Referring to FIG. 6(b), the artificial neural network according to an embodiment of the present invention may include an autoencoder. When the autoencoder inputs original data to the artificial neural network, encodes the data, and reconstructs the encoded data by decoding, the reconstructed data and the input data may have some differences, and the artificial neural network may use these differences. For example, the autoencoder may have a structure in which each of an input layer and an output layer has the same size of 5 and in which a size of a first hidden layer is 3, a size of a second hidden layer is 2, and a size of a third hidden layer is 3, and the number of nodes of the hidden layer gradually decreases as advancing toward an intermediate layer and gradually increases as approaching the output layer. The autoencoder of FIG. 6(b) is an example, and the present invention is not limited thereto. The autoencoder compares an input value of original data with an output value of a reconstructed data, and if a difference between the input value and the output value is large, the autoencoder may determine that the data is not learned, and if a difference between the input value and the output value is small, the autoencoder may determine that the data is pre-learned. Therefore, when the autoencoder is used, reliability of data may be increased. Further, the autoencoder may be used as a compensation means for removing noise of an input signal.

In this case, a mean square error (MSE) may be used for comparing an input value and an output value. As the MSE increases, data may be determined as non-learned data, and as the MSE decreases, data may be determined as pre-learned data.

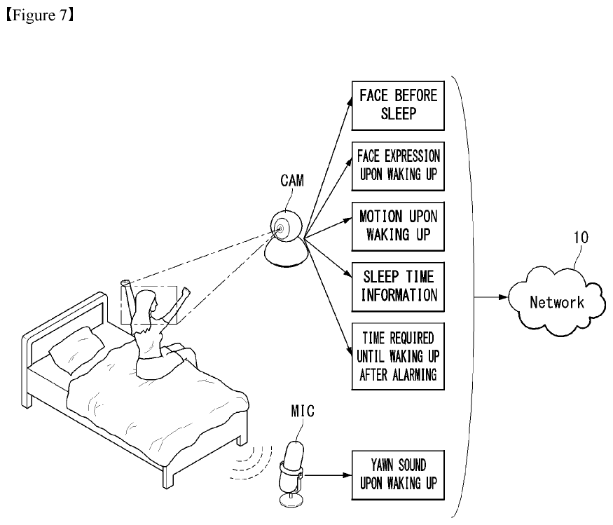

FIG. 7 illustrates a process of obtaining sleep state data from an external terminal according to an embodiment of the present invention.

Referring to FIG. 7, an external monitoring device may include a camera

CAM, a microphone MIC, or the like. Further, the external monitoring device may include not only a camera CAM and a microphone MIC, but also an external terminal having a camera CAM or a microphone MIC. For example, the external terminal may include a smart device such as a smart phone and a smart watch.

The camera CAM may photograph a user to generate image information such as image information and video information including the user. In this case, the image information may include facial recognition information of the user before sleep, facial recognition information upon waking up, motion information upon waking up, sleep time information, time information required until waking up after alarming, and the like. In particular, by checking a condition of the user before sleep, the condition may be used for estimating an appropriate sleep time of the day, and by determining a face state of the user before sleep, the face state may be used for estimating an appropriate sleep time based on flushing or drinking. That is, the present invention has an advantage of estimating an optimal sleep time in consideration of not only a past sleep history but also a condition of the day.

The facial recognition information may include a skin condition of a face, facial expression information, and the like. When the skin condition is less than an average state, an appropriate sleep time may be increased, and even when it is determined that an emotional state of the user is depressed or tired based on the facial expression information, an appropriate sleep time may be increased.

The motion information may include stretching action information and information about a size and the number of tossing and turning. Because a stretching action facilitates blood circulation and improves a physical condition, a day in which stretching is performed in the morning is determined as a day in which the user's condition is relatively good, and thus an appropriate sleep time may be reduced. Further, when tossing and turning in sleep is large or the number of tossing and turning in sleep is a lot, it is determined that the user has not entered deep sleep and thus an appropriate sleep time during next sleep is required to be increased relatively.

The sleep time information may be determined by the difference between a sleep entry time and a wake-up time of an actual user, and a time required until wake-up after alarm may be determined as a measured value of a time required from a time in which alarm of an electronic device having a wake-up alarm function, such as the user's mobile terminal is started until when it is determined that the user wakes up. As a sleep time is extended, fatigue is solved and thus an appropriate sleep time may be reduced, and as a time required to a time determined to fully wake up after wake-up alarm is extended, the user's condition may be determined to be fatigued and thus an appropriate sleep time may be extended.

The microphone MIC may detect a user's voice to generate voice information. In this case, the voice information may include information about a magnitude (dB), a frequency (Hz), and voice recognition duration of the user voice.

The AI server 16 may determine a user's condition based on a magnitude, a frequency, and duration of a voice of the user. Further, the AI server 16 may determine whether a specific voice corresponding to a yawn sound of the user has been detected based on the magnitude, frequency, and duration of the voice. For example, when a voice pattern of the user gradually increases while the user greatly inhales air, rapidly increases once again while the user emits inhaled air, and again gradually decreases after a predetermined time point, and when duration of an overall voice falls within a range of about 5 seconds to 10 seconds, the AI server 16 may determine that the user yawns.

Based on the voice information of the user, when voice information determined to a bad condition due to illness, fatigue or the like is detected or when a yawn sound is detected, the AI server 16 determines that the user state needs rest and thus an appropriate sleep time may be extended.

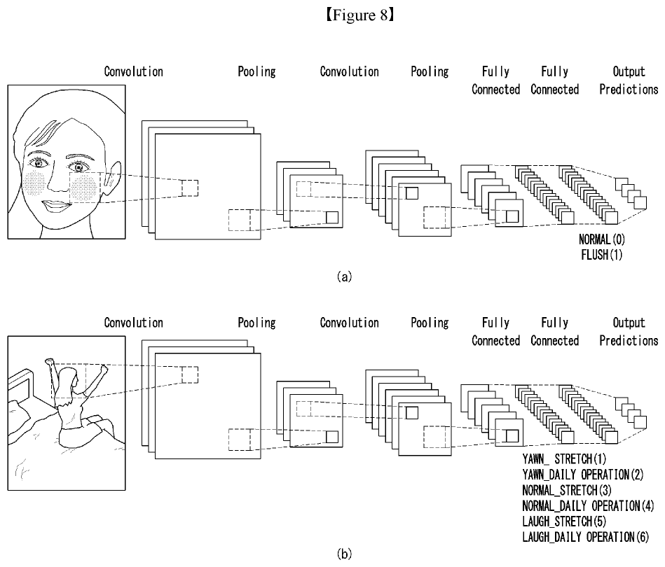

FIG. 8 is a diagram illustrating image processing through CNN according to an embodiment of the present invention.

Referring to FIG. 8, the AI server 16 may recognize an image using a convolutional neural network model. Specifically, the convolutional neural network model uses a local property of image data, and because video data is a sequence of images, a three-dimensional convolutional neural network model may be constructed by adding a time axis as natural extension.

The convolutional neural network model is a kind of a multi-layer artificial neural network model and may effectively recognize data having geometrical relationships between each data dimension, as in images. The convolutional neural network model mitigates complexity of a model by modeling only patterns between dimensions of geometrically close local areas instead of simultaneously modeling all data dimensions.

A convolutional neural network uses a convolution operation and a pooling operation as main elements.

The convolutional operation may indicate that a convolutional neural network models in common in all areas in the form of a kernel instead of independently modeling patterns in each area, and the pooling operation may indicate an operation that enables to counter noise by reducing position information. The pooling operation may reduce a magnitude of the convolution operation by two to three times when a result of the convolution operation is input. In the pooling operation, even if an object moves in parallel in the image, the output value is the same, which enables to be robust to noise.

FIG. 8 illustrates a process of determining a status of a user from image information about the user's face and image information about a motion through a convolution neural network model.

Referring to FIG. 8(a), the AI server 16 may use a convolutional neural network model to distinguish whether the face is a normal state or a flush state from image information of the user's face before sleep. Further, referring to FIG. 8(b), the AI server 16 may determine the user's state upon waking up using the convolutional neural network model, specifically, the user state may include a yawn-stretch state, a yawn-daily operation state, a normal-stretch state, a normal-daily operation state, a laugh-stretch state, a laugh-daily operation state, and the like.

FIGS. 8(a) and 8(b) illustrate that convolution and pulling is repeated twice, but this is merely an example and the number of convolution and pulling is not limited to that shown in the drawing.

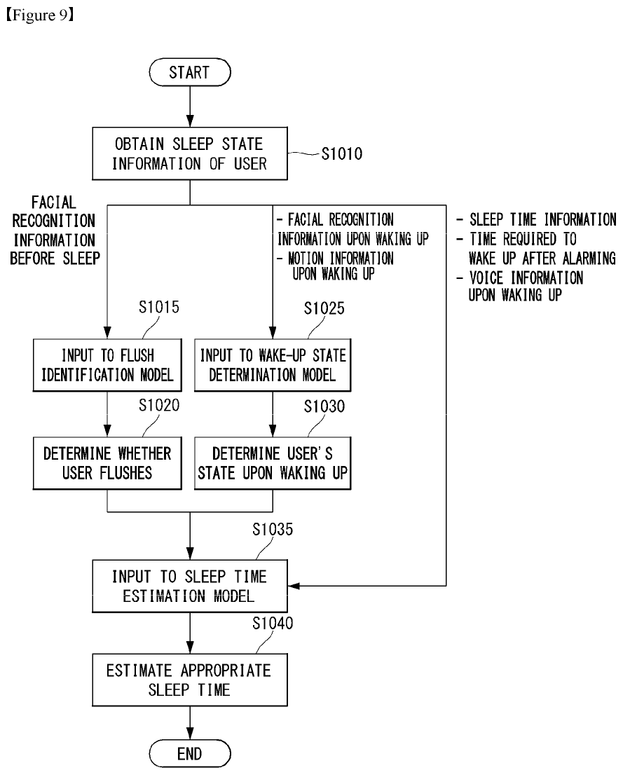

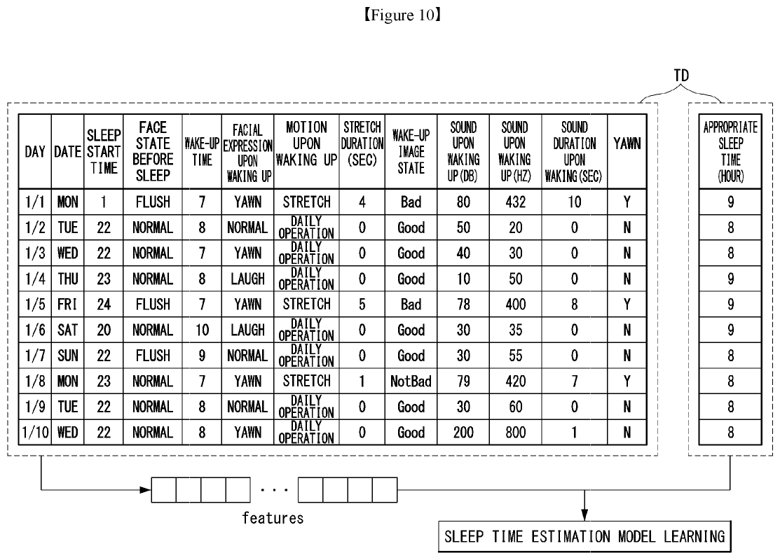

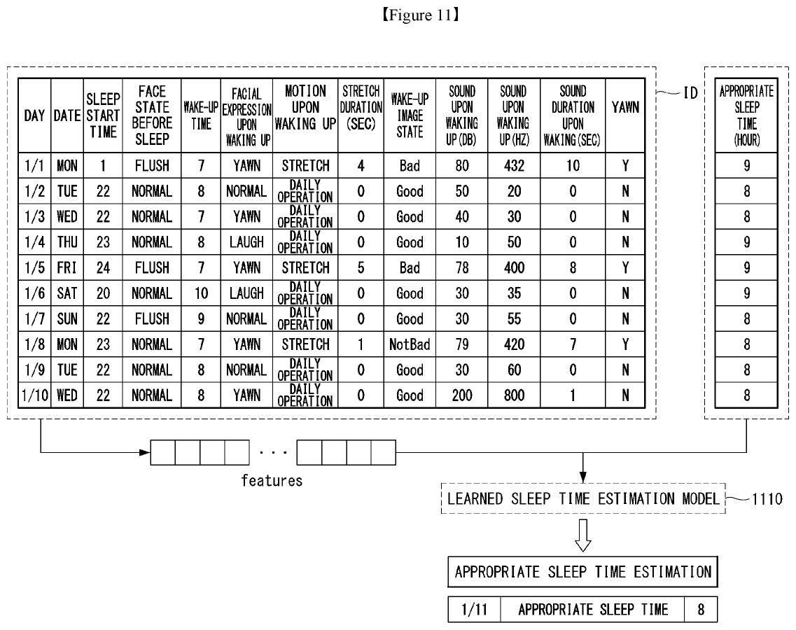

FIG. 9 is a flowchart illustrating a method of estimating an appropriate sleep time according to an embodiment of the present invention, and FIGS. 10 and 11 are diagrams illustrating an example of estimating an appropriate sleep time of a user.

The AI server 16 may receive sleep state data obtained by a camera CAM or a microphone MIC from the network 10 (S1010). As described above, the camera CAM may obtain image information including at least one of facial recognition information before sleep, facial recognition information upon waking up, motion information upon waking up, sleep time information, or time information required to wake up after alarming, and the microphone MIC may include voice information including at least one of sound volume information, frequency information, or duration of a voice. Further, any electronic device equipped with a camera CAM or a microphone as well as a camera CAM and a microphone MIC may be used as information collection means without limitation in a kind.

The AI server 16 may apply facial recognition information before sleep to a flush identification model and determine whether the user flushes according to an output value of the flush identification model (S1015, S1020).

Specifically, facial recognition information of the user before sleep received from the network 10 may be applied to the flush identification model, and it may be determined whether the user flushes from an output value, which is the application result. When the user flushes, the user may be determined to be more tired physically, and flushing may act as an element to extend an appropriate sleep time. Because an appropriate sleep time may be estimated in consideration of the user's state before sleep, there is an advantage of estimating a more accurate sleep time in consideration of not only a past record but also a user's condition immediately before sleep.

The AI server 16 may apply facial recognition information upon waking up or motion information upon waking up to a wake-up state determination model and determine facial expression information or a motion information according to an output value (S1025, S1030). It may be determined whether fatigue is sufficiently solved compared to a sleep time based on a state of a face at the moment when the user wakes up. Specifically, the user's physical condition may be estimated based on a skin condition, flushing, and facial expression of the user's face. Further, it may be determined whether fatigue is solved by the user's behavior upon waking up. For example, it may be determined that the user's fatigue is not sufficiently solved when the user does not wake up on a bed for a predetermined time or falls back to sleep.

Conversely, when the user performs stretching immediately after waking up or immediately performs a daily operation, it may be determined that the user's fatigue is sufficiently solved.

The AI server 16 may apply information on whether a user blushes, facial expression information, motion information, sleep time information, information on a time required to wake up after alarming, or voice information upon waking up to the sleep time estimation model (S1035). In this case, the above-mentioned presence or absence information of flush, facial expression information, motion information, sleep time information, information on a time required to wake up after alarming, and voice information upon waking up may be referred to as factors affecting a sleep time (see FIG. 11).

In this case, the sleep time estimation model may be an artificial neural network model supervision learned by setting factors affecting a sleep time and an appropriate sleep time to learning data TD.

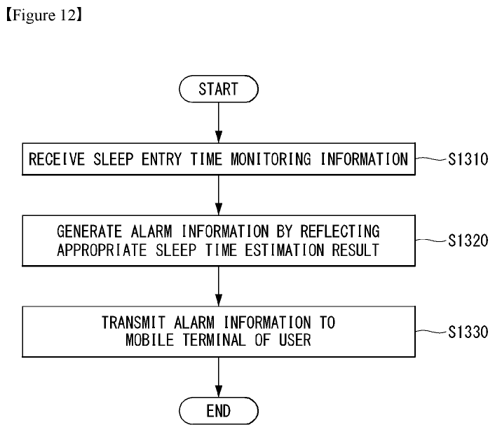

The AI server 16 may determine an appropriate sleep time according to an output value of the sleep time estimation model (S1040). The sleep time estimation model may estimate an appropriate sleep time of a user on a specific date based on the above-described various factors. For example, using a sleep time, a face state before sleep, a wake-up time, a face expression upon waking up, a motion upon waking up, stretch duration, a wake-up image state, loudness upon waking up, a frequency of sound upon waking up, duration of sound upon waking up, and yawning or not measured from January 1 to January 10 as input data ID, an appropriate sleep time on January 11 may be estimated as 8 hours (see FIG. 12)

When the AI server 16 estimates an appropriate sleep time, the AI server 16 may generate and transmit a signal for controlling an electronic device based on the appropriate sleep time. An embodiment thereof will be described later with reference to FIGS. 12 to 15.

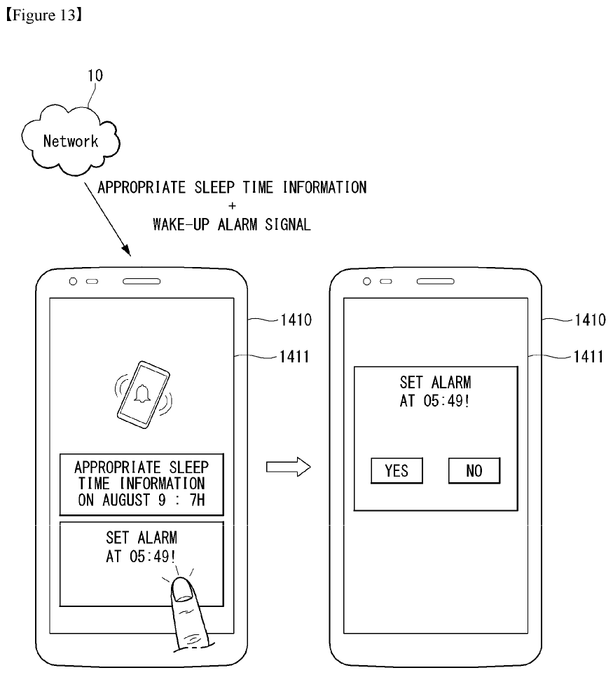

FIGS. 12 and 13 are diagrams illustrating an alarm setting method according to an embodiment of the present invention.

Referring to FIG. 12, the AI server 16 may receive sleep entry time monitoring information from the network 10 (S1310). In this case, the sleep entry time may be estimated from image information or audio information about the user. For example, when a user enters sleep and breathes regularly, and when there is no great tossing and turning, it may be determined that the user enters sleep. For another example, when the user snores or repeats breaths of a predetermined magnitude on a regular basis, it may be determined that the user has entered sleep. For another example, by collecting a bio signal of the user from an electronic device such as a wearable device, it may be determined whether the user enters sleep.

In this case, a sleep entry time of the user may include a real sleep entry time determined that the user has actually entered sleep and an estimated sleep entry time estimated that the user has entered sleep based on deep learning.

By reflecting an appropriate sleep time estimation result, the AI server 16 may generate alarm information (S1320). The AI server 16 may generate a wake-up alarm signal in which the sleep entry time plus the appropriate sleep time is set as a wake-up time.

The AI server 16 may transmit alarm information to the mobile terminal 1410 of the user (S1330). The mobile terminal 1410, having received a wake-up alarm signal from the AI server 16 may display information of the corresponding wake-up alarm through the display 1411. For example, the display 1411 may display information on a specific date, an appropriate sleep time and a wake-up time of the specific date, and an alarm schedule of the wake-up time. In this case, when a touch input is received through the display 1411, a menu for checking a user's intention to an alarm may be additionally displayed (see FIG. 13). The AI server 16 may receive feedback on the appropriate sleep time from the mobile terminal 1410 and reflect the feedback to derivation of a subsequent appropriate sleep time.

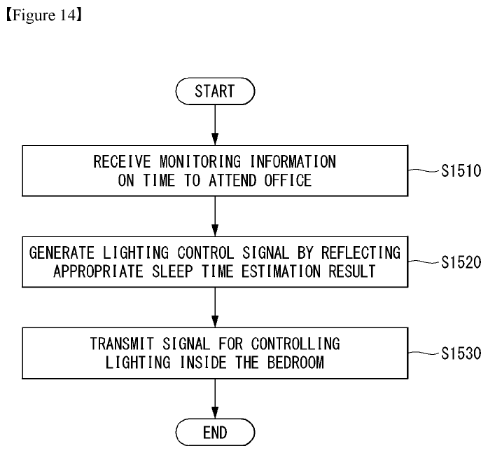

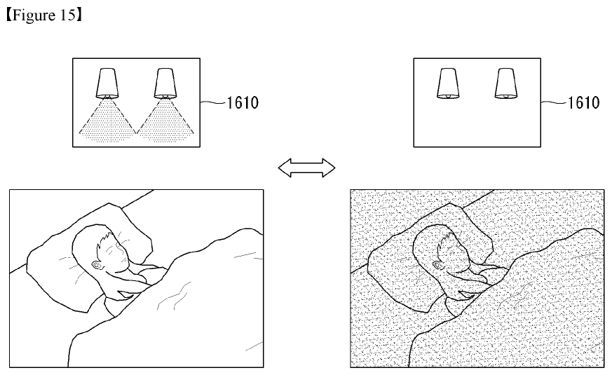

FIGS. 14 and 15 are diagrams illustrating a light control method according to an embodiment of the present invention.

Referring to FIG. 14, the AI server 16 may receive outgoing time information based on an outgoing pattern including a user's attendance pattern from the network 10 (S1510). In this case, the attendance pattern may be derived from movement information of the user, image information obtained from a monitoring device provided in the user's home, and the like. The mobile terminal 1410 of the user or the monitoring device of the user's home may transmit image information, audio information, terminal usage information, etc. to the network 10, and the network 10 may determine an attendance pattern based on the corresponding information. The attendance pattern may be different on a monthly basis, a weekly basis, a daily basis, or a day basis of the week.

By reflecting an appropriate sleep time estimation result, the AI server 16 may generate a lighting 1610 control signal (S1520). Specifically, the AI server 16 may determine the user's scheduled sleep time based on the user's attendance pattern. For example, when the user is scheduled to go to work at 7:00 am and the appropriate sleep time is determined to be 7 hours, the user's bedroom lighting 1610 may be switched to a sleep induction mode or turned off at 00 AM. Specifically, the lighting 1610 may emit light as a wavelength band or illuminance of at least one light, and may induce a person's sleep at a specific color temperature or color wavelength and thus a quality of sleep may be improved using a signal controlling the lighting 1610.

The AI server 16 may transmit a signal for controlling the lighting 1610 inside the bedroom to the lighting 1610 of the user's bedroom (S1530). For example, at the sleep entry time determined based on the appropriate sleep time estimation result, the lighting 1610 receives a control signal from the network 10 and is turned off or switched to a sleep induction mode according to the control signal (see FIG. 14).

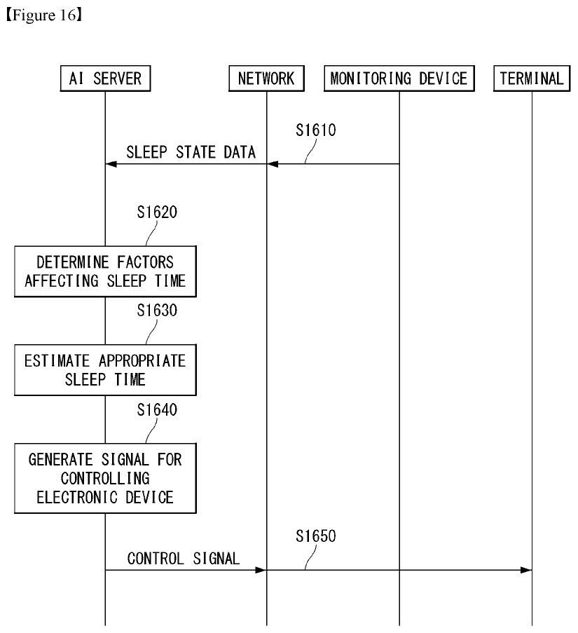

FIG. 16 is an overall sequence diagram according to an embodiment of the present invention.

FIG. 16 is a sequence diagram summarizing the above contents described in FIGS. 9, 12, and 14, and overlapped descriptions will be omitted. Referring to FIG. 16, the AI server 16 may receive sleep state data obtained by the monitoring device from the network 10 (S1610).

The AI server 16 may determine factors affecting a sleep time based on the received sleep state data, and input the factor to a sleep time estimation model to estimate an appropriate sleep time (S1620, 1630).

The AI server 16 may generate a signal for controlling an electronic device including the mobile terminal 1410, the lighting 1610, and the like, based on the appropriate sleep time information, and transmit a control signal to the corresponding electronic device (S1640, S1650).

The present invention may be implemented as a computer readable code in a program recording medium. The computer readable medium includes all kinds of record devices that store data that may be read by a computer system. The computer readable medium may include, for example, a Hard Disk Drive (HDD), a Solid State Disk (SSD), a Silicon Disk Drive (SDD), a read-only memory (ROM), a random-access memory (RAM), a compact disc read-only memory (CD-ROM), a magnetic tape, a floppy disk, an optical data storage device and the like and also include a medium implemented in the form of a carrier wave (e.g., transmission through Internet). Accordingly, the detailed description should not be construed as being limitative from all aspects, but should be construed as being illustrative. The scope of the present invention should be determined by reasonable analysis of the attached claims, and all changes within the equivalent range of the present invention are included in the scope of the present invention.

What is claimed is:

1. A method of analyzing sleep using an AI server, the method comprising:
receiving sleep state data obtained through a monitoring device;
determining factors affecting a sleep time by applying the sleep state data to a previously trained sleep analysis model; and
determining an appropriate sleep time by applying the factors affecting the sleep time to the previously trained sleep time estimation model,
wherein the sleep state data comprise at least one of image information, voice information, or past sleep history information of a user.

2. The method of claim 1, wherein the monitoring device comprises at least one of a camera or a microphone.

3. The method of claim 1, wherein the image information comprises at least one of facial recognition information before sleep, facial recognition information upon waking up, motion information upon waking up, sleep time information, or time information required to wake-up after alarming.

4. The method of claim 1, wherein the voice information comprises at least one of loudness information, frequency information, or duration of a voice.

5. The method of claim 1, wherein the receiving of sleep state data comprises receiving the sleep state data from a 5G network to which the monitoring device is connected.

6. The method of claim 1, wherein the sleep analysis model comprises at least one of a flush identification model or a wake-up state determination model, and
wherein the factors affecting the sleep time comprise at least one of information on whether the user flushes, facial expression information of the user, behavior pattern information of the user, or sound pattern information determined to yawn by the user.

7. The method of claim 6, wherein the determining of factors affecting a sleep time comprises:
applying the sleep state data to the flush identification model; and
determining whether the user flushes according to an output value of the flush identification model.

8. The method of claim 6, wherein the determining of factors affecting a sleep time comprises:
applying the sleep state data to the wake-up state determination model; and
determining the facial expression information or the motion information according to an output value of the wake-up state determination model.

9. The method of claim 1, wherein the determining of an appropriate sleep time comprises:
applying factors affecting the sleep time to the sleep time estimation model; and
determining the appropriate sleep time according to an output value of the sleep time estimation model.

10. The method of claim 9, wherein information about the appropriate sleep time comprises a specific date and the appropriate sleep time of the specific date.

11. The method of claim 1, further comprising generating a signal for controlling an external terminal communicatively connected to the AI server based on the appropriate sleep time.

12. The method of claim 11, wherein the controlling signal is a wake-up alarm signal, and
wherein the generating of a signal comprises:
checking a sleep entry time of a user based on image information obtained through the monitoring device;
determining a wake-up time based on the sleep entry time; and
generating the wake-up alarm signal comprising the wake-up time.

13. The method of claim 11, wherein the controlling signal is a lighting control signal, and
wherein the generating of a signal comprises:
obtaining outgoing time information of the user through the monitoring device;
determining a sleep entry time based on the outgoing time information; and
generating a signal to control at least one of a wavelength or illuminance of light at the sleep entry time.

14. An AI server having a sleep analysis function, the AI server comprising:
a transceiver for receiving sleep state data from an external monitoring device; and
a processor for applying the sleep state data to a previously trained sleep analysis model to determine factors affecting a sleep time, and applying the factors affecting the sleep time to a previously trained sleep time estimation model to determine an appropriate sleep time.

15. The AI server of claim 14, wherein the sleep state data comprise at least one of image information, voice information, or past sleep history information of a user.

16. The AI server of claim 14, wherein the sleep analysis model comprises at least one of a flush identification model or a wake-up state determination model, and
wherein the factors affecting the sleep time comprises at least one of information on whether a user flushes, facial expression information of the user, and motion information of the user.

17. The AI server of claim 16, wherein the processor is configured to:
apply the sleep state data to the flush identification model, and
determine whether the user flushes according to an output value of the flush identification model.

18. The AI server of claim 16, wherein the processor is configured to:
apply the sleep state data to the wake-up state determination model, and
determine the facial expression information or the motion information according to an output value of the wake-up state determination model.

19. The AI server of claim 14, wherein the processor is configured to generate a signal for controlling an external terminal communicatively connected to the AI server based on the appropriate sleep time.

20. The AI server of claim 19, wherein the controlling signal comprises any one of a wake-up alarm signal and a lighting control signal.
